(19) United States
(12) Patent Application Publication
(10) Pub. No.: US 2025/0017447 A1
(43) Pub. Date: Jan. 16, 2025

(54) ENDOSCOPE HAVING AN ELEMENT POSITIONED IN A BEAM PATH, ENDOSCOPE ARRANGEMENT, METHOD FOR PROVIDING INFORMATION AND/OR FOR IDENTIFYING AN ENDOSCOPE, USE OF AN ELEMENT POSITIONED IN A BEAM PATH OF AN ENDOSCOPE, ELEMENT

(71) Applicant: Schölly Fiberoptic GmbH, Denzlingen (DE)

(72) Inventors: Markus GERN, Emmendingen (DE); Christoph RAPP, Freiburg (DE); Markus DEBATIN, Freiburg (DE); Pascal STRIEBEL, Denzlingen (DE); Michael SCHWÄRZLE, Denzlingen (DE)

(73) Assignee: Schölly Fiberoptic GmbH, Denzlingen (DE)

(21) Appl. No.: 18/766,830

(22) Filed: Jul. 9, 2024

(30) Foreign Application Priority Data
Jul. 11, 2023 (DE) 102023118298.6

(51) Int. Cl. A61B 1/00; A61B 1/002; A61B 1/04; A61B 90/92; A61B 90/94

(52) U.S. Cl. CPC A61B 1/00059; A61B 1/00004; A61B 1/00163; A61B 1/002; A61B 1/042; A61B 90/92; A61B 90/94

(57) ABSTRACT

An element positioned in a beam path of an endoscope is proposed, which adjoins an element-free area to the beam passage. The element is made planar having various surface areas, and a label and/or coding is formed via at least two different coefficients of transmission of the different surface areas of the element, which are in turn different from a coefficient of transmission in the element-free area. The label and/or coding contains a unique identifier of the endoscope, via which identification of the endoscope is enabled and/or information of an endoscope is provided. The label and/or coding is captured, recognized, and/or processed by a camera during a setting of the camera, in particular during a white balance of the camera.

### CROSS-REFERENCE TO RELATED APPLICATIONS

This application claims priority from German Patent Application No. DE 10 2023 118 298.6, filed Jul. 11, 2023, which is incorporated herein by reference as if fully set forth.

### TECHNICAL FIELD

The invention relates to an endoscope having an element positioned in a beam path, which adjoins an element-free area to the beam passage, wherein the element is made planar having various surface areas.

The invention additionally relates to a method for providing information and/or for identifying an endoscope having a camera, in particular a camera, and an element positioned in a beam path, which adjoins an element-free area to the beam passage, wherein the element is made planar having various surface areas.

The invention finally relates to a use of an element positioned in a beam path, which adjoins an element-free area to the beam passage, wherein the element is made planar having various surface areas.

### BACKGROUND

Providing diaphragms of an endoscope with markings, which are generated, for example, via perforated discs or are made visible by means of fluorescence methods, in order to be able to identify the endoscope, is known from practice, for example.

### SUMMARY

The invention is therefore based on the object of enabling an identification and/or a provision of information of an endoscope by means of a camera and an element positioned in a beam path of the endoscope, wherein the element is made planar having various surface areas, in that, via at least two different coefficients of transmission of the surface areas, a label and/or coding containing a unique identifier of the endoscope and/or optical properties of the camera is formed, which is recognized and evaluated by the camera, in particular automatically.

According to the invention, one or more of the features disclosed herein are provided to achieve the stated object. In particular, to achieve the stated object according to the invention, it is therefore proposed, in an endoscope having an element positioned in a beam path, that the surface areas have at least two different coefficients of transmission, wherein the at least two different coefficients of transmission are different from a coefficient of transmission in the element-free area.

The element-free area can be formed, for example, by an optical aperture to the beam passage.

The element-free area can, for example, be free of optical elements and/or can have a coefficient of transmission of air.

The at least two different coefficients of transmission of the different surface areas of the element can be generated, for example, by an imprint which varies in the different surface areas of the element, for example, is chromatic or achromatic and/or has wavelength-dependent or wavelength-independent transmission differences.

It can therefore be provided, for example, that a relatively dark, for example a black, imprint is applied to a transparent and/or semitransparent carrier material of the element. Furthermore, vice versa, it can be provided, for example, that a correspondingly adversative imprint is applied to a dark, for example a black, carrier material.

It is particularly advantageous here that, for example, the beam path can be deliberately influenced via the different coefficients of transmission of the element that are thus generated.

Moreover, it is advantageous that the at least two different coefficients of transmission of the surface areas of the element can therefore be generated in an uncomplicated and cost-effective manner.

In a further advantageous embodiment, it can be provided that a label and/or coding is formed via the at least two different coefficients of transmission of the element.

It can be provided here, for example, that the coding is designed, for example, as a light-dark coding and/or a color coding having, for example, complementary colors or the like. The coding can have, for example, a wavelength-dependent transmission and/or wavelength-dependent coefficients of transmission, in particular in nonvisible wavelength ranges. This can also be provided accordingly for the label, for example.

The label and/or coding can therefore particularly advantageously be designed so that they can be perceived without problems, for example optically, by a camera having an image processing system, a character recognition system, and/or a user.

In particular, the label and/or coding can contain an identifier of the endoscope.

It is particularly advantageous here that the label and/or coding can therefore be used, for example, for verifying and/or validating the respective endoscope, and a use, for example, of an endoscope which is unsuitable for carrying out an endoscopic method can be prevented.

It can therefore also advantageously be determined, for example, whether the endoscope to be used meets various requirements.

In particular, the label and/or coding can contain optical properties of a camera.

The optical properties can be, for example, color corrections and/or image corrections of the camera, which, for example, can be automatically loaded into the camera and used therein.

It is particularly advantageous here that therefore, for example in the determination of the oxygen content of blood, in which it is of elementary importance to detect the accurate color value, possible distortions of the color value of the blood, which can arise, for example, owing to the optical arrangement of the endoscope, can be automatically compensated and therefore prevented.

The label and/or coding can in particular be damped in relation to the free beam path.

Therefore, for example, the occurrence of undesired scattered light can particularly advantageously be prevented and, for example, an impairment of the visible image in normal operation of the endoscope can be minimized.

In a further advantageous embodiment, it can be provided that the at least two different coefficients of transmission are wavelength-dependent.

It is particularly advantageous here that the at least two different coefficients of transmission can therefore be generated in an uncomplicated manner, for example by color distinctions, for example, via a or the above-mentioned light-dark imprint.

In a further advantageous embodiment, it can be provided that the element positioned in the beam path is a diaphragm, preferably a field diaphragm.

It is particularly advantageous here that therefore, for example, positioning of the element in the area on an image plane can be enabled and in particular the label and/or coding can be read and/or recognized without problems.

In a further advantageous embodiment, it can be provided that the element-free area is a field of view which determines an image size.

It is particularly advantageous here that, for example, a change of the image sharpness, which can be adapted, for example, for better representation of the label and/or coding that can be formed via the at least two different coefficients of transmission, has no influence on the image size.

In a further advantageous embodiment, it can be provided that the element having the at least two different coefficients of transmission is formed in an image plane, in particular a last image plane.

It is particularly advantageous here that the label and/or coding can be sharply imaged and/or digitally extracted, for example, by the or a camera, for example the above-mentioned camera, in particular without changing a previously set focus.

In a further advantageous embodiment, it can be provided that the coefficient of transmission is selected to be at least 50% compared to the free beam path.

It is particularly advantageous here that therefore, for example, a light component which can pass through the at least two different coefficients of transmission of the planar element, in particular in normal operation of the endoscope, can be neglected.

The coefficient of transmission is preferably selected in a range from 0.1% to 10% in relation to the free beam path.

The risk of scattered light, which at worst could result in corruption of the endoscopic findings in normal operation, for example, can therefore particularly advantageously be reduced to a minimum.

In a further advantageous embodiment, it can be provided that the endoscope has a rigid shaft, wherein at least two rod lenses are formed in the rigid shaft.

One advantage of the rigid shaft in relation to a flexible shaft is that, for example, a camera can be decoupled, in particular to use the endoscope with the human eye.

In a further advantageous embodiment, it can be provided that the endoscope is connectable to a camera.

It can be provided in this case, for example, that the image recorded by the camera is depicted, for example, on a display and/or monitor.

Furthermore, it can be provided, for example, that the camera is connectable to a camera control unit, by which signals can advantageously be processed.

The generated images from the endoscope can therefore moreover particularly advantageously be digitally represented, for example.

In particular, it can be provided that an eyepiece of the endoscope is connectable to a camera.

The eyepiece particularly advantageously enables, for example, a problem-free change between a digital endoscopic observation and an endoscopic observation using the human eye.

In particular, the camera has an image processing system and/or a character recognition system.

It is particularly favorable here that via the image processing system and/or the character recognition system, for example, an automatic recognition of the or a label and/or coding, for example a QR code, which can preferably be generated via the at least two different coefficients of transmission on the planar element, can be automatically captured, read, evaluated, and/or processed.

Alternatively or additionally, it can be provided that the endoscope, in particular an eyepiece of the endoscope, is connectable to a camera to form an endoscope arrangement, comprising the endoscope and the camera.

Therefore, for example, carrying out an endoscopic intervention can particularly advantageously be facilitated.

In a further advantageous embodiment, it can be provided that the label and/or coding becomes visible upon an overexposure.

It can be provided here, for example, that the overexposure takes place during a setting of the camera, for example a white balance.

The at least two different coefficients of transmission accordingly are particularly advantageously preferably selected so that it is possible to prevent impairment of the endoscopic image, in particular during normal operation of the endoscope.

Alternatively or additionally, the features of the co-independent claim, directed to an endoscope arrangement having an endoscope and a camera, are provided to achieve the stated object according to the invention.

The advantages already described, which can be implemented, for example, by connecting the endoscope to the camera, can particularly advantageously be generated using the endoscope arrangement.

Alternatively or additionally, to achieve the stated object according to the invention, the features of the co-independent claim, which is directed to a method for providing information and/or for identifying an endoscope, in particular an above-mentioned endoscope, which adjoins an element-free area to the beam passage, wherein the element is made planar having various surface areas, are provided. In particular, it is therefore proposed, to achieve the stated object according to the invention, that, in particular before normal operation of the endoscope, a label and/or coding that can be formed via at least two different coefficients of transmission of the surface areas is used for identifying the endoscope.

Therefore, for example, it can additionally advantageously be determined, via the at least two different coefficients of transmission of the surface areas of the element and the label and/or coding that can be formed thereby, which endoscope is used.

The label and/or coding can be, for example, a QR code that can be formed by the at least two coefficients of transmission, a light-dark coding, and/or a color coding, which can be made visible by an overexposure, for example.

All further embodiments of the label and/or coding which can be formed via the at least two different coefficients of transmission are also conceivable.

The label and/or coding can preferably contain a unique identifier of the endoscope.

The camera usable for the respective endoscope can therefore advantageously be selected, for example.

In a further advantageous embodiment, it can be provided that the label and/or coding is captured and processed by the camera.

This can be implemented, for example, via an image processing system, a character recognition system, and/or a camera control unit. Further camera systems which are designed to capture, process, read, and/or evaluate labels and/or codings in particular are also conceivable.

It is particularly favorable if the corresponding label and/or coding is, for example, automatically recognized by the camera and the correspondingly read and/or evaluated information is preferably communicated to an operator.

In a further advantageous embodiment, it can be provided that optical properties of the camera are captured and processed through the label and/or coding.

Therefore, for example, color corrections and/or image corrections can particularly advantageously be automatically loaded into the camera and used therein.

Moreover, it is extremely advantageous in this case that therefore in particular erroneous findings due, for example, to color casts of the optical arrangement of the endoscope, which can in turn corrupt an actual color value, for example during a measurement of the oxygen content of blood, can be prevented.

In a further advantageous embodiment, it can be provided that the camera is connected to the endoscope, in particular an eyepiece of the endoscope.

Therefore, for example, a digital endoscopic observation and an endoscopic observation using the human eye can advantageously be implemented.

In a further advantageous embodiment, it can be provided that the identification of the endoscope is carried out before a use of the endoscope.

It is particularly advantageous here that, for example, it can be ensured that the elementary information of the respective endoscope for carrying out an endoscopic intervention, which is necessarily to be established before the intervention, can be provided and/or displayed.

Therefore, for example, above-mentioned color casts of the or an optical arrangement of the endoscope, which can result, for example, in corruption of the actual color value and therefore possibly in a corrupted endoscopic finding, can also advantageously be recognized by the identification of the endoscope before the intervention and in particular the camera can be set accordingly.

Alternatively or additionally, it can be provided in a further advantageous embodiment that the identification of the endoscope is carried out during a setting of the camera, preferably during a white balance.

The white balance can be implemented here, for example, by an overexposure. During the setting of the camera, a short-term overexposure can also be performed immediately before and/or after the white balance.

The at least two coefficients of transmission of the surface areas of the element can particularly advantageously preferably be selected to be small enough that the label and/or coding that can be formed in this way becomes visible only when such an overexposure takes place. Therefore, for example, an excessive beam passage at the label and/or coding, which is in turn undesirable in normal operation of the endoscope and increases the risk of scattered light, can also advantageously be prevented.

Furthermore, it is advantageous that in this way, for example, a double check of the endoscope, namely with respect to the color adjustment and the identification of the respective endoscope, can be implemented in one step. It is therefore particularly favorable that due to an identification of the endoscope during the white balance, for example, no additional method step is necessary for validating and/or verifying the endoscope.

In a further advantageous embodiment, it can be provided that a signal is output after an identification of the label and/or coding.

The signal can be, for example, an acoustic and/or optical signal, preferably a digital signal. It can accordingly be provided, for example, that a successful recognition and/or evaluation of the label and/or coding outputs, for example, a corresponding tone in order to communicate to a user, for example, that a successful identification of the endoscope has taken place.

Furthermore, it can be provided, for example, that the various items of information of the endoscope about the label and/or coding for identifying the endoscope are automatically evaluated by the camera, for example, due to which in turn, for example, a signal generated in this way is processed by means of digital signal processing and the various items of information of the endoscope are preferably displayed optically, for example on a display.

It is therefore particularly advantageous that it can be displayed and/or communicated directly to a user if the respective endoscope and/or the or a camera cannot and/or should not be used to carry out the endoscopic intervention, for example, on the basis of the input information.

In a further advantageous embodiment, it can be provided that the label and/or coding is digitally hidden by the camera in a normal operation of the endoscope, in particular by a digital circular diaphragm.

Therefore, for example, optically interfering factors of the label and/or coding, which is marginally visible in normal operation, can particularly advantageously be completely eliminated.

In a further advantageous embodiment, it can be provided that the camera, in particular in the case of an autofocus system, initially moves to a position so that the label and/or coding is sharply imaged.

It is particularly favorable if such a focusing of the label and/or coding takes place simultaneously to the or a white balance, in particular the above-mentioned white balance, since in this way defocusing of the element-free area outside the or a normal operation can be carried out without problems.

Alternatively or additionally, to achieve the stated object according to the invention, the features of the co-independent claim directed to a use of an element positioned in a beam path of an endoscope, which adjoins an element-free area to the beam passage, wherein the element is made planar having various surface areas, are provided. In particular, it is therefore proposed according to the invention, to achieve the stated object, that a label and/or coding for identifying the endoscope, in particular in a color spectrum, can be formed by at least two different coefficients of transmission of the surface areas of the element.

It can be provided here, for example, that the label and/or coding for identifying the endoscope is automatically captured, read, evaluated, and/or processed by a camera.

An erroneous use of an endoscope unsuitable for an endoscopic intervention and/or a camera unsuitable for the respective endoscope, for example, can therefore particularly advantageously be prevented.

Alternatively or additionally, it can be provided according to the invention that a label and/or coding for providing information of the endoscope can be formed by at least two different coefficients of transmission of the surface areas of the element.

The information can contain here, for example, optical properties of the or a camera preferably connectable to the endoscope, which can advantageously be automatically loaded directly into the camera and used therein, for example.

Alternatively or additionally, to achieve the stated object according to the invention, the features of the co-independent claim, directed to an element which is positioned in a beam path of an endoscope, wherein the element adjoins an element-free area to a beam passage, wherein the element is made planar having various surface areas, are provided. In particular, it is therefore proposed, to achieve the stated object according to the invention, that the element is multilayered, in particular two-layered, wherein a second layer having a different coefficient of transmission in comparison to the first layer is applied to a first transparent layer to form a label and/or coding on the element.

The second layer can be, for example, a dark film here, for example to generate a coefficient of transmission different from the transparent first layer, which film can be applied, preferably printed, onto the first transparent layer, for example. It is particularly favorable if the second film has, for example, specific openings, via which, for example in combination with the transparent first layer, the label and/or coding can be formed, in particular via a pattern. A QR code can therefore be generated, for example.

Information of the endoscope, for example, can therefore particularly advantageously be read and/or evaluated by a camera connectable to the endoscope, in particular by means of an image processing system and/or character recognition system. Furthermore, for example, an identification of the endoscope can also advantageously be implemented, due to which it is also in turn favorable that therefore a verification and/or validation of the endoscope can be enabled.

Furthermore, it can be provided, for example, that a constant beam path over an entire surface of the element is generated at the label and/or coding. This can be achieved, for example, in that the coefficients of transmission of the different surface areas have a coefficient of transmission gradient over the entire surface of the element, for example, from the element-free area adjoining the beam passage to an outer (edge) area of the element extending radially outward.

It can therefore advantageously be achieved, for example, that the label and/or coding can be adapted to a spatial beam intensity via the coefficients of transmission. Moreover, it is advantageous that the label and/or coding can preferably be perceived uniformly over the entire surface of the element.

Alternatively or additionally, it can be provided according to the invention that the first layer is semitransparent.

Therefore, for example, the occurrence of scattered light can particularly advantageously be minimized.

In a further advantageous embodiment, it can be provided that the element is a diaphragm, in particular a field diaphragm.

The element can therefore particularly advantageously be positioned, for example, in an image plane of the endoscope, due to which the label and/or coding, for example, can be sharply imaged by the or a camera and/or can be read without problems by a user.

### BRIEF DESCRIPTION OF THE DRAWINGS

The invention will now be described in more detail on the basis of exemplary embodiments, but is not restricted to the exemplary embodiments. Further exemplary embodiments result by a combination of the features of individual or multiple claims with one another and/or with individual or multiple features of the exemplary embodiment.

In the figures, each in a greatly simplified representation

FIG. 1 shows a two-dimensional sectional representation of an endoscope in a side view having an element positioned in a beam path,

FIG. 2 shows a two-dimensional schematic structure of an endoscope having a camera,

FIG. 3 shows a two-dimensional schematic representation of a section of an element positioned in a beam path of an endoscope in a top view having at least two different coefficients of transmission, via which a label and/or coding is formed.

### DETAILED DESCRIPTION

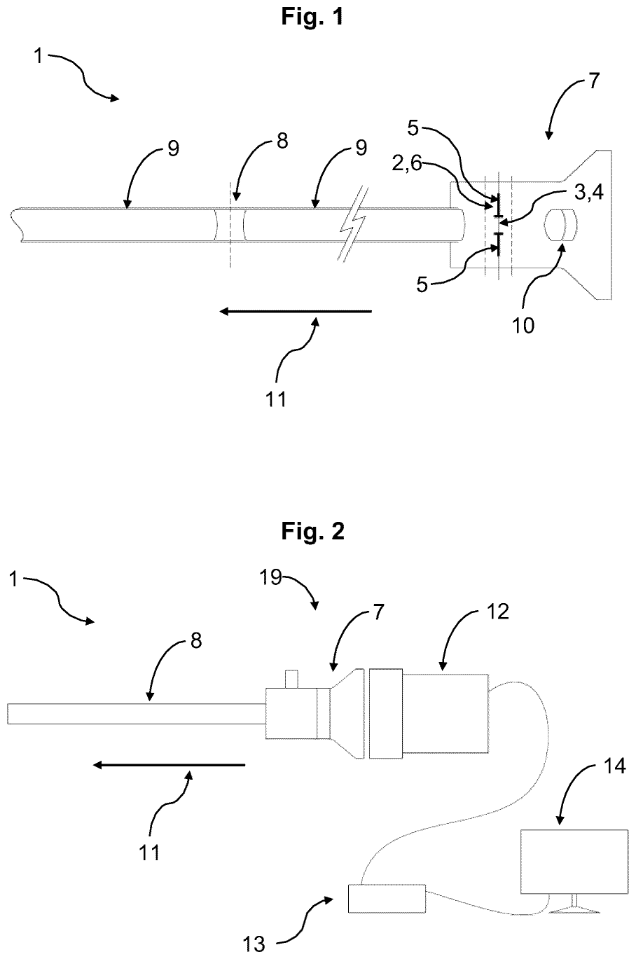

FIG. 1 shows an endoscope, identified as a whole by 1, in a sectional representation having an element 2 positioned in a beam path, which adjoins an element-free area 3 to the beam passage.

The element 2 is made planar having various surface areas 5. In this exemplary embodiment, the element 2 is a field diaphragm 6, which is positioned in an eyepiece 7 of the endoscope 1 in a last image plane. The element-free area 3 of the element 2 forms a field of view 4, via which an image size is determined.

Furthermore, it is evident that the endoscope 1 has a rigid shaft 8, wherein two rod lenses 9 are formed in the rigid shaft 8.

The element 2 is furthermore formed in this exemplary embodiment after a concavely and convexly shaped eyepiece lens 10 in the viewing direction 11.

The exemplary embodiment according to FIG. 2 differs from the preceding exemplary embodiment in that the eyepiece 7 of the endoscope 1 is connected to a camera 12 to form an endoscope arrangement 19. Components and functional units which are equivalent or identical functionally and/or structurally to the preceding exemplary embodiment are identified by the same reference numerals and are not described separately once again.

Furthermore, it is evident that the camera 12 is connected to a camera control unit 13 and the camera control unit 13 is in turn connected to a display 14. The camera 12 is moreover formed having an image processing and character recognition system.

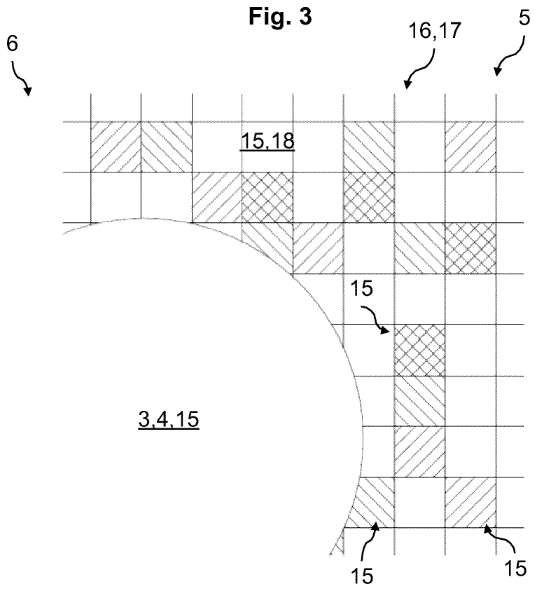

FIG. 3 shows an exemplary embodiment of an element 2, positioned in a beam path of an endoscope 1, having the various surface areas 5 and the element-free area/field of view 3, 4. It can be seen that the surface areas 5 of the element 2 have different coefficients of transmission 15, wherein more than two different coefficients of transmission 15 of the surface areas 5 are different from a coefficient of transmission 15 in the element-free area 3.

The respective surface areas 5 in the figure are shaded differently to represent different coefficients of transmission 15.

Furthermore, it can be seen that a label 16 and coding 17 is formed via the different coefficients of transmission 15 of the element 2. This is implemented in this exemplary embodiment in that a further, second layer is applied to a first transparent layer 18 of the element 2.

In a further exemplary embodiment (not shown), the first layer is made semitransparent.

The label 16 and coding 17, which is formed via the different coefficients of transmission 15 on the surface areas 5 of the element 2, contains in this exemplary embodiment an identifier of a or the endoscope 1 and optical properties of a or the camera 12.

If the element 2 shown in this exemplary embodiment is used as a field diaphragm 6 in the endoscope 1 shown according to FIG. 2, the label 16 and coding 17 is captured and processed by the camera 12 during a setting of the camera 12 in the context of a white balance and therefore before a usage of the endoscope 1, and the endoscope 1 is therefore identified and information is provided.

As soon as the endoscope 1 has been identified, a signal is output, which is in turn processed by the camera control unit 13 (cf. FIG. 2).

The label 16 and coding 17 is subsequently digitally hidden in normal operation of the endoscope 1 by a circular diaphragm (not shown in this exemplary embodiment).

According to the invention, an element 2 positioned in a beam path of an endoscope 1 is therefore proposed, which adjoins an element-free area 3 to the beam passage, wherein the element 2 is made planar having various surface areas 5, wherein via at least two different coefficients of transmission 15 of the different surface areas 5 of the element 2, which are in turn different from a coefficient of transmission 15 in the element-free area 3, a label 16 and/or coding 17 is formed, wherein the label 16 and/or coding 17 contains a unique identifier of the endoscope 1, via which an identification of the respective endoscope 1 is enabled and/or information of the endoscope 1 is provided, wherein the label 16 and/or coding 17 is automatically captured, recognized, and/or processed by a camera 12, preferably during a setting of the camera 12, in particular during a white balance of the camera 12.

### LIST OF REFERENCE NUMERALS

1 endoscope
2 element
3 element-free area
4 field of view
5 surface areas
6 field diaphragm
7 eyepiece
8 rigid shaft
9 rod lens
10 eyepiece lens
11 viewing direction
12 camera
13 camera control unit
14 display
15 coefficient of transmission
16 label
17 coding
18 transparent layer
19 endoscope arrangement

1. An endoscope (1), comprising:
an element (2) positioned in a beam path, which adjoins an element-free area (3) to the beam path, the element (2) is planar and includes various surface areas (5), the surface areas (5) have at least two different coefficients of transmission (15), and the at least two different coefficients of transmission (15) are different from a coefficient of transmission (15) in the element-free area (3).

2. The endoscope (1) according to claim 1, further comprising at least one of a label (16) or coding (17), which contains at least one of an identifier of the endoscope (1) or optical properties of a camera (12), and which is damped in relation to the element-free area of the beam path, is formed via the at least two different coefficients of transmission (15) of the element (2).

3. The endoscope (1) according to claim 1, wherein the at least two different coefficients of transmission (15) are wavelength-dependent.

4. The endoscope (1) according to claim 1, wherein the element (2) positioned in the beam path is a diaphragm (6).

5. The endoscope (1) according to claim 1, wherein the element-free area (3) is a field of view (4), which determines an image size.

6. The endoscope (1) according to claim 1, wherein the element (2) having the at least two different coefficients of transmission (15) is formed in an image plane.

7. The endoscope (1) according to claim 1, wherein the coefficient of transmission (15) is selected to be least 50% compared to the free beam path.

8. The endoscope (1) according to claim 1, further comprising a rigid shaft (8), and at least two rod lenses (9) are in the rigid shaft (8).

9. The endoscope (1) according to claim 1, wherein the endoscope (1) is connectable to a camera (12), with the camera (12) having at least one of an image processing system or a character recognition system.

10. The endoscope (1) according to claim 1, wherein the at least one of the label (16) or the coding (17) becomes visible upon an overexposure.

11. An endoscope arrangement (19) comprising the endoscope (1) according to claim 1 and a camera (12) connected to the endoscope, the camera (12) having at least one of an image processing system or a character recognition system.

12. A method for at least one of providing information or for identifying an endoscope (1) having a camera (12) and an element (2) positioned in a beam path, which adjoins an element-free area (3) to the beam passage, wherein the element (3) is made planar having various surface areas (5), the method comprising:
before normal operation of the endoscope (1), using at least one of a label (16) or coding (17) formed via at least two different coefficients of transmission (15) of the various surface areas (5), and which contain at least one of a unique identifier of the endoscope (1) or the information, to at least one of identify the endoscope (1) or the information.

13. The method according to claim 12, further comprising capturing and processing the at least one of the label (16) or the coding (17) by the camera (12).

14. The method according to claim 12, wherein optical properties of the camera (12) are captured and processed through the at least one of the label (16) or the coding (17).

15. The method according to claim 12, further comprising connecting the camera to an eyepiece of the endoscope (1).

16. The method according to claim 12, further comprising carrying out an identification of the endoscope (1) before a usage of the endoscope (1), during a setting of the camera (12), and/or during a white balance.

17. The method according to claim 12, further comprising outputting a signal after an identification of at least one of the label (16) or the coding (17).

18. The method according to claim 12, wherein the at least one of the label (16) or the coding (17) is digitally hidden by the camera (12) in normal operation of the endoscope (1) by a digital circular diaphragm.

19. The method according to claim 12, further comprising initially moving the camera (12) to a position so that the at least one of the label (16) or the coding (17) is sharply imaged.

20. The method of claim 12, wherein the at least one of the label (16) or the coding (17) for at least one of identifying or providing information of the endoscope (1), in a color spectrum, is formed by the at least two different coefficients of transmission (15) of the surface areas (5) of the element (2).

21. An element (2), which is positioned in a beam path of an endoscope (1) and is adapted to be positioned in an element-free area (3) to a beam passage, the element (2) comprising:
at least two planar layers having various surface areas (5), a second one of the layers, having a different coefficient of transmission (15) in comparison to a first one of the layers, is applied to a first transparent (18) or semitransparent layer to form at least one of a label (16) or coding (17) on the element (2).

22. The element (2) according to claim 21, wherein the element (2) is a diaphragm (6).
